(19) United States
(12) Patent Application Publication
(10) Pub. No.: US 2023/0285023 A1
(43) Pub. Date: Sep. 14, 2023

(54) Fistula Formation Devices And Methods

(71) Applicant: Bard Peripheral Vascular, Inc., Franklin Lakes, NJ (US)

(72) Inventors: Andrzej J. Chanduszko, Chandler, AZ (US); Michael Adam Randall, Gilbert, AZ (US)

(73) Assignee: Bard Peripheral Vascular, Inc., Franklin Lakes, NJ (US)

(21) Appl. No.: 18/041,340

(22) PCT Filed: Aug. 14, 2020
(86) PCT No.: PCT/US2020/046343

§ 371 Date: Feb. 10, 2023

(51) Int. Cl. A61B 17/11

(52) U.S. Cl. CPC A61B 17/11; A61B 2017/1107; A61B 2017/1139

(57) ABSTRACT

A fistula formation device includes a first engagement member and a second engagement member. The first engagement member is configured to be positioned within a first vessel. The first engagement member includes a plate defining a first opening therethrough. The second engagement member is configured to be positioned within a second vessel. The second engagement member includes a base plate having a second opening extending therethrough, and one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

### TECHNICAL FIELD

The present specification generally relates to fistula formation devices and methods and, more specifically, fistula formation devices and methods including first and second engagement members operable to form a fistula between first and second vessels.

### BACKGROUND

A fistula is a passageway formed between two internal organs within a body. For example, forming a fistula between two blood vessels can have one or more beneficial functions. The formation of a fistula between an artery and a vein may provide access to the vasculature for hemodialysis patients. Specifically, forming a fistula between an artery and a vein (also known as an AV fistula) allows blood to flow quickly between the vessels while bypassing the capillaries. Needles, catheters, and/or other cannulas may then be inserted into the blood vessels near the fistula to draw blood from the circulatory system, pass it through a dialysis machine, and return it to the body. The quickened flow provided by the fistula may provide for effective hemodialysis. In a mature fistula, the flow rate through the fistula may be on the order of 300-500 ml/min, or may be on the order of 300-1500 ml/min, or more.

However, the surgical procedures conventionally used for fistula formation may be very invasive.

### SUMMARY

As noted above, one challenging aspect of fistula formation is that conventional procedures may be highly invasive. Accordingly, embodiments, provided herein are directed to minimally invasive devices, systems, and methods for forming a fistula.

In one embodiment, a fistula formation device includes a first engagement member and a second engagement member. The first engagement member is configured to be positioned within a first vessel. The first engagement member includes a plate defining a first opening therethrough. The second engagement member is configured to be positioned within a second vessel. The second engagement member includes a base plate having a second opening extending therethrough, and one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

In another embodiment, a system for forming a fistula comprises one or more sheaths configured to be advanced through at least one of a first vessel and a second vessel. The fistula formation device is delivered to the first vessel and the second vessel with the one or more sheaths. The fistula formation device includes a first engagement member configured to be positioned within the first vessel and a second engagement member configured to be positioned within the second vessel. The first engagement member includes a plate defining a first opening therethrough. The second engagement member includes a base plate having a second opening extending therethrough, and one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

In yet another embodiment, a method of forming a fistula includes advancing one or more sheaths into at least one of a first vessel and a second vessel, deploying a first engagement member within the first vessel, and deploying a second engagement member within the second vessel. The first engagement member includes a plate defining a first opening therethrough. The second engagement member includes a base plate having a second opening extending therethrough, and one or more retention members extending from the base plate around the second opening, the method further includes coupling the first engagement member to the second engagement member by inserting the one or more retention members of the second engagement member through the plate of the first engagement member to secure the first engagement member to the second engagement member, thereby forming an open conduit through the first engagement member and the second engagement member between the first vessel and the second vessel.

In yet another embodiment, a fistula formation device incudes a first magnetic disk and a second magnetic disk. The first magnetic disk is configured to be positioned within a first vessel, the first magnetic disk defining a first opening therethrough. The second magnetic disk is configured to be positioned within a second vessel, the second magnetic disk defining a second opening therethrough. Each of the first and second magnetic disks are magnetically attracted to one another such that the first opening is axially aligned with the second opening thereby providing a fluid passage between the first vessel and the second vessel. At least one of the first magnetic disk and the second magnetic disk comprise one or more spacing elements mounted to the at least one of the first magnetic disk and the second magnetic disk and configured to maintain a separation distance of the first magnetic disk from the second magnetic disk.

In yet another embodiment, a method of forming a fistula includes deploying a first engagement member within a first vessel, the first engagement member comprising a first magnetic disk defining a first opening therethrough, and deploying a second engagement member within a second vessel, the second engagement member comprising a second magnetic disk defining a second opening therethrough. Each of the first and second magnetic disks are magnetically attracted to one another such that the first opening is axially aligned with the second opening thereby providing a fluid passage between the first vessel and the second vessel. At least one of the first magnetic disk and the second magnetic disk comprise one or more spacing elements mounted to the at least one of the first magnetic disk and the second magnetic disk and configured to maintain a separation distance of the first magnetic disk from the second magnetic disk.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a fistula formation device including a first engagement member and a second engagement member, according to one or more embodiments shown and described herein;

FIG. 1B schematically depicts a first sheath being advanced through a first vessel and a cutting element being advanced from the first sheath from the first vessel and into the second vessel, according to one or more embodiments shown and described herein depicts;

FIG. 1C schematically depicts a second sheath being advanced over a guidewire extending from the first sheath of FIG. 1B to a position within the second vessel, according to one or more embodiments shown and described herein;

FIG. 1D schematically depicts the first engagement member of the fistula formation device being advanced from the first sheath and the second engagement member of the fistula formation device being advanced from the second sheath of FIG. 2B, according to one or more embodiments shown and described herein;

FIG. 1E schematically depicts formation of a fistula between the first engagement member the second engagement member of the fistula formation device of FIG. 1D, according to one or more embodiments shown and described herein;

FIG. 2A schematically depicts an alternative delivery assembly for a fistula formation device, according to one or more embodiments shown and described herein;

FIG. 2B schematically depicts the delivery assembly of FIG. 2A being advanced through a first vessel, according to one or more embodiments shown and described herein;

FIG. 2C schematically depicts formation of a fistula between the first vessel and the second vessel with the fistula formation device of FIG. 2A, according to one or more embodiments shown and described herein;

FIG. 3A schematically depicts another embodiment of a fistula formation device including a first engagement member and a second engagement member, according to one or more embodiments shown and described herein;

FIG. 3B schematically depicts delivery the first engagement member, as illustrated in FIG. 3A, from a first vessel and into a second vessel, according to one or more embodiments shown and described herein;

FIG. 3C schematically depicts delivery of the second engagement member of the fistula formation device of FIG. 3A in to the first vessel, according to one or more embodiments shown and described herein;

FIG. 3D schematically depicts formation of the fistula with the first and second engagement members of the fistula formation device of FIG. 3A, according to one or more embodiments shown and described herein;

FIG. 4A schematically depicts a top view of a first engagement member and a second engagement member of a fistula formation device unassembled from one another, according to one or more embodiments shown and described herein;

FIG. 4B schematically depicts a side view of the first engagement member and the second engagement member of FIG. 4A, according to one or more embodiments shown and described herein;

FIG. 4C schematically depicts a delivery configuration of the first engagement member and the second engagement member of FIG. 4A, according to one or more embodiments shown and described herein;

FIG. 4D schematically illustrates a cross-section of the fistula formation device of FIGS. 4A-4C mounted within the vasculature of a user, according to one or more embodiments shown and described herein;

FIG. 5A schematically depicts another embodiment of a fistula formation device including a first magnetic ring and a second magnetic ring, according to one or more embodiments shown and described herein;

FIG. schematically 5B depicts yet another embodiments of a fistula formation device including a first engagement member and a second engagement member, according to one or more embodiments shown and described herein; and

FIG. 6 depicts a method for forming a fistula using a fistula formation device according to one or more embodiments shown and described herein.

### DETAILED DESCRIPTION

Embodiments of the present disclosure are directed to devices, systems, and methods, configured to form fistulas with minimal surgical invasiveness. Moreover, embodiments of the present disclosure are directed to forming fistulas with improved patency and structural integrity. For example, devices and methods as provided herein provide an implantable device which may be delivered through a patient's body passages (e.g., vasculature) and used to form a fistula between the body passages. Accordingly, a fistula may be formed with minimal surgical invasiveness in the body. In one embodiment a device for forming a fistula includes a first engagement member and a second engagement member. The first and second engagement members, when coupled to one another, may form and/or maintain corresponding openings formed within a first vessel and a second vessel to provide fluid communication between the first vessel and the second vessel. For example, the first engagement member may be deployed in one vessel and the second engagement member may deployed in an adjacent vessel. The first engagement member and the second engagement member may be drawn toward and coupled to one another such the openings of the first engagement member and the second engagement member are axially aligned and the openings within the first and second vessels come together thereby facilitating formation of a fluid passage (i.e., fistula) between the first vessel and a second vessel. Various embodiments of the fistula formation devices, systems, and methods will be described in more detail herein.

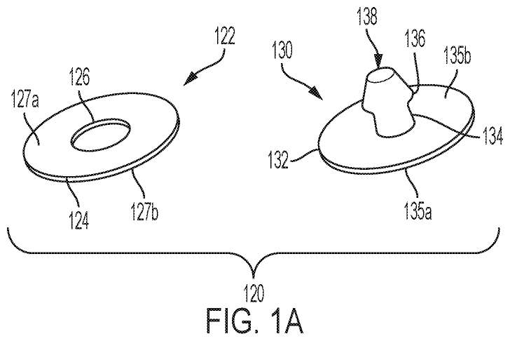

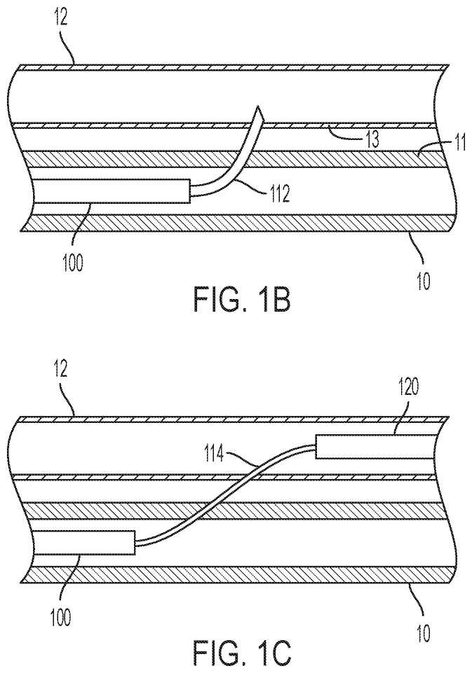

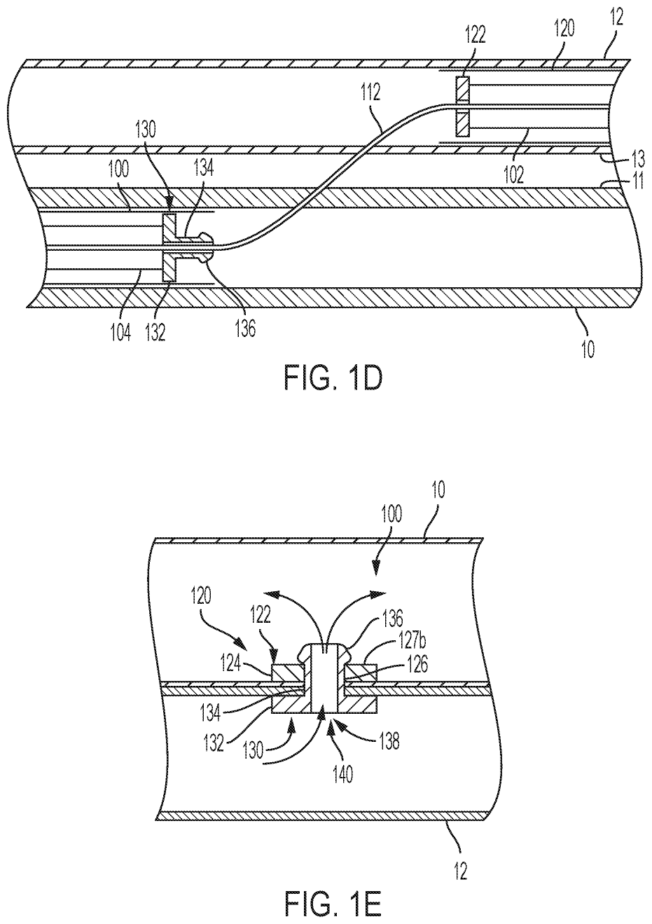

FIG. 1A schematically illustrates an unassembled fistula formation device 120. The fistula formation device 120 includes a first engagement member 122 and a second engagement member 130. As will be described in greater detail below, the first and second engagement members 122, 130 may be joined together within a body of a patient to form a fistula between a first vessel 10 and a second vessel 12, such as illustrated in FIG. 1E. As an example, a vessel may refer to any hollow structure within the body (e.g., veins, arteries, organs, etc.).

The first engagement member 122 is sized and configured to be positioned within a first vessel 10 and includes a plate 124 defining a first opening 126 extending therethrough. The plate 124 may define a first surface 127a and a second surface 127b opposite the first surface 127a, wherein spacing between the first surface 127a and the second surface 127b defines a plate thickness. The first surface 127a and the second surface 127b may be parallel and planar. At least one of the first surface 127a and the second surface 127b is configured to engage a side wall of a vessel. The plate 124 may be thin such that a thickness of the plate 124 is less than about 0.3 mm, less than about 0.2 mm, less than about 0.1 mm, etc. The first engagement member 122 is illustrated as being generally oval-shaped; however, the first engagement member 122 may be round, square-shaped, rectangular, or any other shape. Additionally, the first opening 126 may also be oval-shaped as depicted or some other shape (e.g., square-shaped, round, rectangular, etc.). As will be described in greater detail below the first engagement member 122 is configured to retain the second engagement member 130 to impinge vessel wall tissue between one of the first surface 127a and the second surface 127b of the first engagement member 122 and the second engagement member 130.

The second engagement member 130 generally includes a base plate 132 having a second opening 138 extending therethrough and one or more retention members 134 surrounding at least a portion of the second opening 138.

The base plate 132 may define a first surface 135a and a second surface 135b opposite the first surface 135a, wherein spacing between the first surface 135a and the second surface 135b defines a base plate thickness. The first surface 135a and the second surface 135b may be parallel and planar. The base plate thickness may be thin such as less than about 0.3 mm, less than about 0.2 mm, less than about 0.1 mm. etc. The base plate 132 is illustrated as being generally oval-shaped; however, the base plate 132 may be round, square-shaped, rectangular, or any other shape. It is noted that the second opening 138 may be round as depicted or some other desired shape (e.g., square-shaped, oval-shaped, rectangular, etc.).

The second surface 135b may be configured to engage and side wall of a vessel and may include one or more retention members 134 that may extend from the second surface 135b. In the illustrated embodiment, the one or more retention members 134 include a single retention member 134 that completely encircles the opening 138 so as to provide a conduit from the base plate 132 to a distal end of the retention member 134. As will be described below, the conduit may extend through the walls of the first and second vessels 10, 12 to provide a fluid passage from one vessel to the other.

Toward the distal end of the retention member 134 may be formed one or more retention tabs 136. A retention tab 136 may include any structure which is configured and shaped to engage the first engagement member 122 to couple to first engagement member 122 to the second engagement member 130. In the illustrated embodiment, the retention tab 136 is formed along the entire outer perimeter of the retention member 134, such that the retention member 134 has an increased diameter at the retention tab 136. In some embodiments, the retention tab 136 may not be continuous around the perimeter but may be discontinuous such that there are a plurality of retention tabs 136 positioned on and spaced around the retention member 134.

FIGS. 1B-1E illustrate an embodiment of a method of delivery of the fistula formation device 120 into a body of a patient and formation of a fistula between a first vessel 10 and a second vessel 12. The first and second vessels 10, 12 of the patient may include any two adjacent vessels (e.g., a vein and an artery, a vein and a vein, an artery and an artery, or any other body vessel or cavity combination) between which it may be desirable to form a fistula. As illustrated in FIG. 1B, the first vessel 10 may have a sidewall 11 which may be adjacent a sidewall 13 of the second vessel 12.

As will be described, various delivery elements combined with the fistula formation device 120 may form a system for forming a fistula. For example, the system may include one or more sheaths configured to be advanced through at least one of the first vessel 10 and the second vessel 12. In the illustrated embodiment, a first sheath 100 may be advanced through the first vessel 10. The first sheath 100 may define a lumen through which various portions of the fistula formation device 120 and or other system elements may be advanced for delivery.

In various embodiments, a cutting element such as a needle 112 may be advance from the first sheath 100 to cross between the first vessel 10 and into a second vessel 12. Stated another way, the needle 112 may cut an opening through the sidewall 11 of the first vessel 10 and then another opening through the sidewall 13 of the second vessel 12. Other cutting elements may include, but are not limited to, knives, electrodes, wires, or the like.

The needle 112 may be nitinol, stainless steel, or the like. In some embodiments, the needle 112 may be made from a shape memory material that allows the needle 112 to bend as it is advanced from the sheath 100 such that the needle 112 is directed to pierce the sidewall 11 of the first vessel 10 and enter the sidewall 13 of the second vessel 12. As noted above, by crossing the needle 112 from the first vessel 10 and into a second vessel 12 an opening may be formed within both the side wall 11 of the first vessel 10 and the sidewall 13 of the second vessel 12. As will be described, the opening formed within the first and second vessels 10, 12 may be brought together to form a fistula between the first vessel 10 and the second vessel 12.

The needle 112 may also define a lumen through which a guidewire 114 may be advanced to cross from the first vessel 10 and into the second vessel 12, as illustrated in FIG. 1C. The guidewire 114 may be advanced through the second vessel 12, into a second sheath 116 also defining a lumen, and out of the body of the patient. The guidewire 114, as will be further described, provides a structure along which the first and second engagement members 122, 130 may be advanced as illustrated in FIGS. 1D-1E to couple the first engagement member 122 to the second engagement member 130 for fistula formation.

FIG. 1D schematically illustrates a cross sectional view of the first sheath 100 and the second sheath 116 within the first vessel 10 and the second vessel 12, respectively. In the illustrated embodiment, the second engagement member 130 is positioned within the first sheath 100 and the first engagement member 122 is positioned within the second sheath 116. However, the first engagement member 122 may instead be positioned within the first sheath 100 and the second engagement member 130 may be positioned within the second sheath 116. Each of the first engagement member 122 and the second engagement member 130 are sized so as to be able to travel axially through the first and second sheaths 100, 116 over the guidewire 114.

To advance the first and second engagement member 122, 130 to the desired location, such that the first and second engagement members 122, 130 may be used to form a fistula, each of the first and second engagement members 122, 130 may be mounted to a pusher 102, 104. The pusher 102, 104 may be a wire, rod, tube, or other structure mounted to the first and/or second engagements members 122, 130 to allow a physician to guide the first and second engagement members 122, 130 through the respective sheaths 100, 116 and into engagement with one another. Once the first and second engagement members 122, 130 are coupled to one another, the pushers 102, 104 may be removed, as illustrated in FIG. 1E. For example, the pushers 102, 104 may snap or twist off the first and second engagement members 122, 130 under certain or predetermined applications of force. In some embodiments, and as illustrated, the plate 124 of the first engagement member 122 and the based plate 132 of the second engagement member 130 may be mounted to an end of the pushers 102, 104 so as to be oriented perpendicularly to an elongate axis of the pushers 102, 104. However, in some embodiments, the plate 124 of the first engagement member 122 and the base plate 132 of the second engagement member 130 may be mounted to an end of the pushers 102, 104 such as to be oriented at some oblique angle relative to the elongate axis of the pushers 102, 104. An oblique angle may aid in directing the first engagement member 122 and the second engagement member 130 toward one another for coupling the first engagement member 122 to the second engagement member 130 across the sidewalls 11, 13 of the first vessel 10 and the second vessel 12.

Using the pushers 102, 104, for example, the retention member 134 of the second engagement member 130 may be inserted through the openings made within the first vessel 10 and the second vessel 12 (e.g., by the needle or other cutting element) into the first engagement member 122 thereby coupling the first engagement member 122 to the second engagement member 130. It is noted that vessel tissue of the first vessel 10 and the second vessel 12 may be elastic such that it is able stretch and retract around the retention member 134 as it is passed through the sidewalls 11, 13 or the first vessel 10 and the second vessel 12 to seal against the retention member 134. For example, FIG. 1E illustrates a final assembly of the first engagement member 122 coupled with the second engagement member 130 such that a fistula is formed between the first vessel 10 and the second vessel 12. As illustrated, the retention member 134 of the second engagement member 130 has been pushed through both the sidewall 11 of the first vessel 10 and the sidewall 13 and the second vessel 12 such that the retention tab 136 is positioned within the second vessel 12. The first engagement member 122 is pushed over the retention tab 136 of the retention member 134 such that the retention tab 136 extends over the second surface 127b (e.g., engages the second surface 127b) of the plate 124 of the first engagement member 122 and the retention member 134 is positioned within the first opening 126 of the first engagement member 122. Accordingly, sidewall tissue of the first vessel 10 and the second vessel 12 is impinged between the plate 124 of the first engagement member 122 and the base plate 132 of the second engagement member 130. When assembled, the first and second openings 126, 138 may define a flow passage 140 from the first vessel 10 and the second vessel 12 to allow bodily fluid to flow from the first vessel 10 and into the second vessel 12 or vice versa. In particular, in the present embodiment, the retention member 134, which is seated within the first opening 126 of the first engagement member 122, defines the flow passage 140 from the first vessel 10 and the second vessel 12

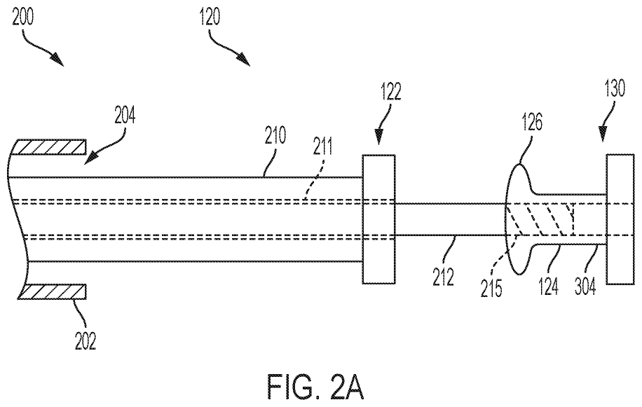

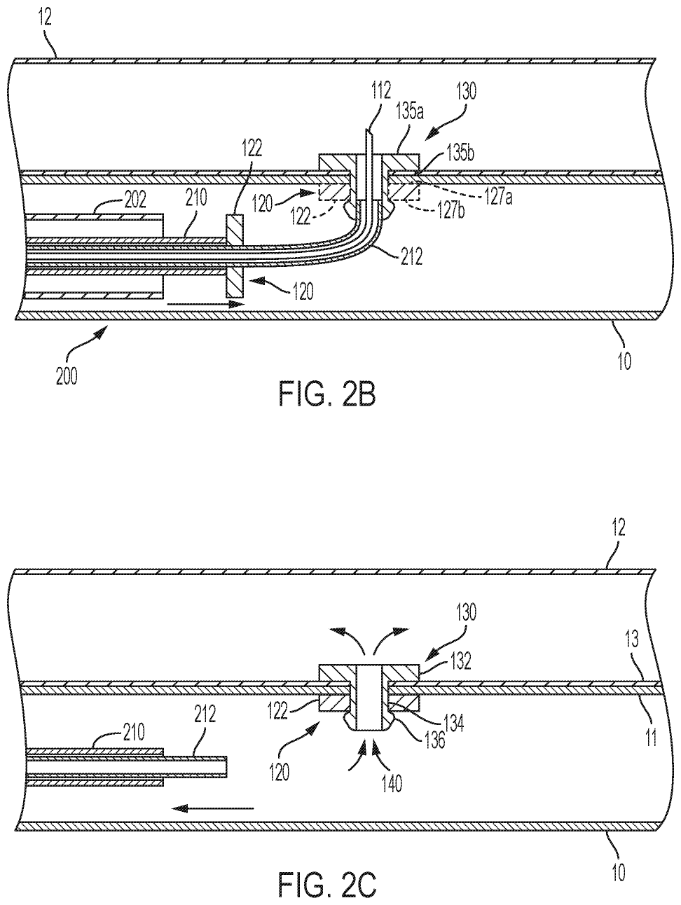

FIG. 2A depicts an alternative delivery system 200 including a single sheath 202 used to deliver and assemble the fistula formation device 120. Single sheath delivery methods may have the added benefit of only needing a single access point in either vessel to form a fistula versus two access points in a dual sheath delivery method. The sheath 202 defines a lumen 204 through which the first engagement member 122 and the second engagement member 130 may be advanced. For example, the first engagement member 122 may be mounted to a first pusher 210, such as described above. The first pusher 210 may be a tube defining a lumen 211 extending therethrough. Arranged concentrically within the first pusher 210 may be a second pusher 212 mounted to the second engagement member 130. For example, the second engagement member 130 may be threadingly engaged with the second pusher 212. For example, the second engagement member 130 may include internal threads along the retention member 134 that engages with external threads formed on a distal end of the second pusher 212 to form a threaded engagement 215. The second pusher 212 may also define a lumen (not shown) therethrough, to allow for passage of a guidewire, needle, or other cutting element (not shown). The first and second pushers 210, 212 may be flexible and/or biased to allow assembly of the fistula formation device 120 through the sidewall 11, 13 of the first and second vessels 10, 12.

FIGS. 2B and 2C generally illustrate formation of a fistula using the delivery system 200. In particular, a crossing between the first vessel 10 to the second vessel 12 may be achieved by advancing of a cutting element (e.g., needle 112) and/or a guidewire from the first vessel 10 to the second vessel 12. The second pusher 212 may then be advanced to push the second engagement member 130 through the openings formed in the first vessel 10 and the second vessel 12, such that the base plate 132 of the second engagement member 130 is positioned within the second vessel 12 and the retention tab 136 is positioned within the first vessel 10. For example, the vessel tissue of the first vessel 10 and the second vessel 12 may be elastic (as noted above) such that the openings formed in the first vessel 10 and the second vessel 12 may be stretched over the retention tab 136 and retract around the retention member as 134 as the retention member 134 is advanced through the vessel tissue. With the second pusher 212 remaining attached to the second engagement member 130, the first pusher 210 may be advanced over the second pusher 212 to push the first engagement member 122 over the end of the retention member 134. For example, and as illustrated in FIG. 2C, the first engagement member 122 is pushed over the one or more retention tabs 136, such that the one or more retention tabs 136 extend over the second surface 127b of the first engagement member 122 and couple the first engagement member 122 to the second engagement member 130. Accordingly, and as described above, tissue of the first vessel 10 and the second vessel 12 is impinged between the first engagement member 122 and the second engagement member 130, and the first formation device defines a flow path 140 between the first vessel 10 and the second vessel 12.

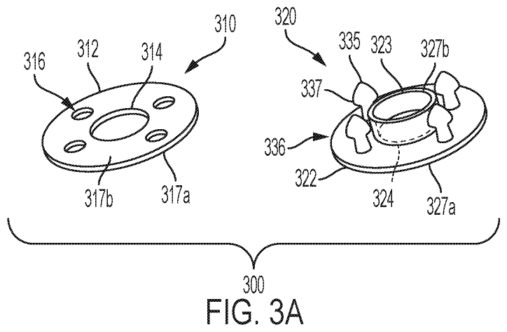

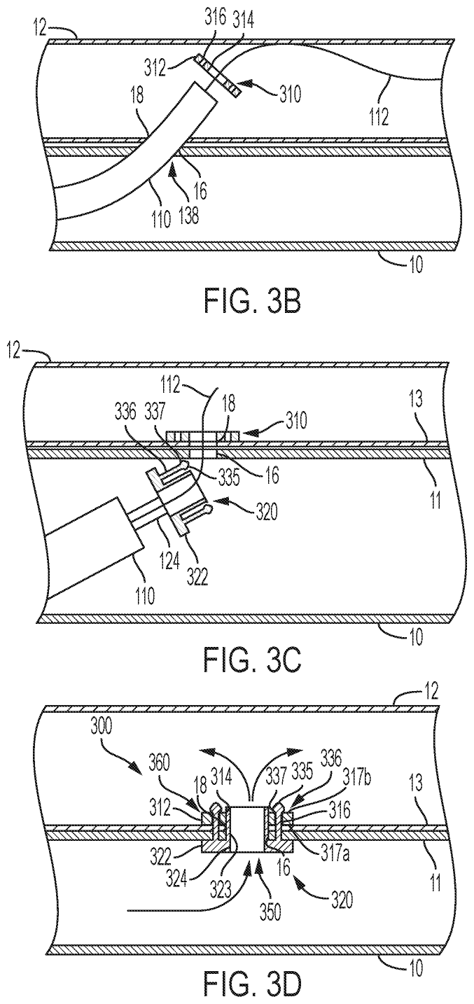

FIG. 3A depicts another embodiments of a fistula formation device 300. The fistula formation device 300 is similar to the fistula formation device 120 described above, except for the noted or otherwise apparent differences. Moreover, delivery systems and methods for delivery of the fistula formation device 120 are the same or similar to those described above with respect to the fistula formation device 120.

The fistula formation device 300 includes a first engagement member 310 and a second engagement member 320. Similar to the above, the first engagement member 310 includes a plate 312 with a first opening 314 extending therethrough. In this embodiment, a plurality of retention openings 316 (e.g., two or more retention openings, three or more retention openings, four or more retention openings, etc.) are positioned radially around the first opening 314 so as to be axially offset from the first opening 314. The retention openings 316 may have any shape such, but not limited to, circular, oval-shaped, elongated, curved, or any polygonal or non-polygonal shape. In embodiments, elongated retention openings may aid in alignment with retention members of the second engagement member 320, described below.

The second engagement member 320 includes a base plate 322 and a second opening 324 formed therein. A conduit 323 may extend from the base plate 322 and encircle the opening 324. As with embodiments above, the first opening 314 of the first engagement member 310 may be aligned with the second opening 324 of the second engagement member 320 to allow for formation of a fluid flow path. For example, the conduit 323 may be positioned within the first opening 314 of the first retention member 2310 to provide the flow path. However, in the present embodiment, a plurality of retention members 336 are positioned radially around the second opening 324 and/or the conduit 323 and spaced therefrom. The plurality of retention members 336 may be projections that extend from a second surface 327b (opposite first surface 327a) of the base plate 322. The plurality of retention members 336 may include two or more retention members 336, three or more retention members 336, four or more retention members 336, etc. to match the number of retention openings 316 formed in the first engagement member 310. Each of the plurality of retention members 134 may include sharp distal tips 335 configured to pierce vessel tissue.

Each of the plurality of retention members 336 may also include a retention tab 337, which are configured to couple the first engagement member 310 to the second engagement member 320. For example, the plurality of retention members 336 are configured to be advanced through the plurality of retention openings 316 of the first engagement member 310. The retention tab 337 of each retention member 336 may engage or extend over the first surface 317a or the second surface 317b of the first engagement member 310 to couple the first engagement member 310 to the second engagement member 320.

While the embodiment illustrated in FIG. 3A may be delivered and implanted within the first and second vessels 10, 12, as described above, FIGS. 3B-3D illustrate another example for delivery a fistula formation device, which may be used with any of the embodiments described herein. In the illustrated embodiment, a single sheath 110 may be passed through an opening 16 formed in the first vessel 10 and another opening 18 formed in the second vessel 12. As noted above, vessel tissue may be elastic so as to deform to allow passage of the single sheath 110. The openings 16, 18 may be formed with any cutting device such as a needle, knife, electrode, etc. The first engagement member 310 may be deployed from the sheath 110 in the second vessel 12. For example, a pusher (not shown), such has described above, may push the first engagement member 310 from the sheath 110 into the second vessel 12. Referring now the FIG. 3C, the sheath 110 may be withdrawn out of the second vessel 12 and into the first vessel 10. Removal of the sheath 110 may allow the openings 16, 18 within the vessel tissue to retract in size. The first engagement member 310 may be pulled into engagement, by the pusher (not shown), into contact with the sidewall 13 of the second vessel 12. In another embodiment a guidewire 114 may be removably coupled to the first engagement member 310 and may act as a tether allowing an operator to pull the first engagement member 310 into contact with the sidewall 13 of the second vessel 12 for assembly with the second engagement member 320. In yet further embodiments, a second sheath (not shown) positioned within the second vessel 12, may be used to push the first engagement into contact with the sidewall 13 of the second vessel 12 for assembly with the second engagement member 320.

A pusher 102 may then be extended out of the sheath 110 to position the second engagement member 320 with the first vessel 10. The pusher 102 may cause the plurality of retention members 336 to engage and pierce the tissue of the first vessel 10 and the second vessel 12 and extend through the plurality of retention openings 316 of the first engagement member 310. Once pushed through the plurality of retention openings 316, as illustrated in FIG. 3D, the plurality of retention tabs 337 may engage and/or extend over the second surface 317b of the first engagement member 310 thereby coupling the first engagement member 310 to the second engagement member 320 and impinging the vessel sidewall tissue of the first and second vessels 10, 12 between the first engagement member 310 and the second engagement member 130. The openings 16, 18 of the first and second vessels 10, 12 may be aligned with the first opening 314 of the first engagement member 310 and the second opening 324 of the second engagement member 320 such that a flow passage 350 is formed and maintained by the first engagement member 310 and the second engagement member 320 thereby creating a fistula between the first vessel 10 and the second vessel 12. For example, the conduit 323 of the second engagement member 320 may be inserted through the openings 16, 18 of the first vessel 10 and the second vessel 12, and into the first opening 314 of the first engagement member simultaneously as the plurality of retention members 336 extend through the plurality of retention opening 316. Accordingly, the conduit 323 may maintain fluid communication between the first vessel 10 and the second vessel 12.

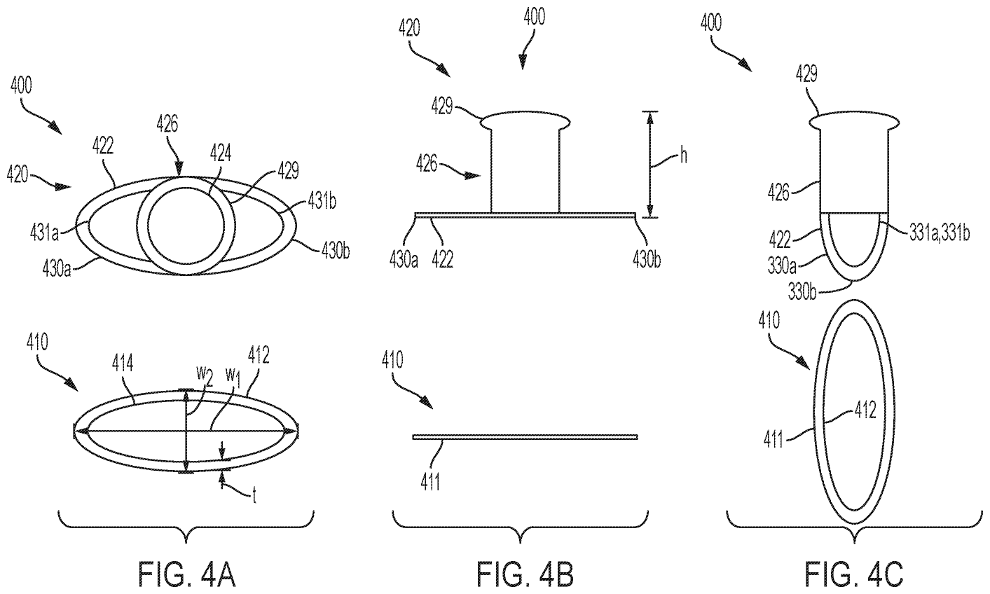

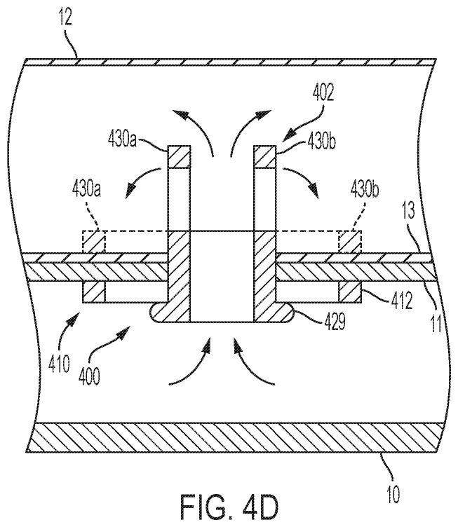

FIGS. 4A-4D illustrate yet another embodiment of a fistula formation device 400. As with the above, embodiments, the fistula formation device 400 includes a first engagement member 410 and a second engagement member 420. FIG. 4A illustrates a top view of the fistula formation device 400 in an unassembled configuration, FIG. 4B illustrates a side view of the fistula formation device 400 in the unassembled configuration, and FIG. 4C illustrates a delivery configuration of the unassembled fistula formation device 400.

As in embodiments described above, the first engagement member 410 may include a plate 412. However, in this embodiment, the plate 412 may be oblong and the first opening 414 formed through the plate 412 may also be oblong. Accordingly, the plate 412 may generally comprise an oblong ring of material having a wall thickness, t, of, for example, about 0.1 mm to about 0.3 mm. The plate 412 may have a first width, w₁, of about 4 mm to about 7 mm, for example. The plate 412 may have a second width, w₂, of about 1 mm to about 3 mm, for example. The plate 412 may be relatively thin and less than, for example 1 mm.

The second engagement member 420 may include a base plate 422 similar in size and shape to the plate 412 of the first engagement member 410. In particular, the base plate 422 may be oblong and have a second opening 424 extending therethrough. In the present embodiment, a retention member 426 is attached to the base plate 422 and surrounds the second opening 424 providing a conduit through the second engagement member 420. Similar to embodiments above, the retention member 426 may include one or more retention tabs 429 extending from an end of the retention member 426 and configured to engage the first engagement member 410. The second engagement member 420 may include an overall height, h, of about 1 mm to about 5 mm, though larger or smaller dimensions are contemplated and possible.

Formed within the base plate 422 on either side of the retention member 426 may be one or more cut outs 431a, 431b such that the base plate 422 defines flexible wings 430a, 430b. The flexible wings 430a may be foldable in a delivery configuration as illustrated in FIG. 4C. In the delivery configuration, the flexible wings 430a may be folded so as to align with an elongate axis of the retention member 426. The flexible wings 430a may be biased (e.g., spring biased) to the open position illustrated in FIG. 4A. In some embodiments, more than two wings may be included (e.g., three wings, four wings, five wings, etc.), without departing from the scope of the present disclosure.

Referring now to FIG. 4D, during deployment, the second engagement member 420 may positioned within the first and second vessels 10, 12 by any of the methods described above. For example, the base plate 422 may be folded in the delivery position as illustrated in FIG. 4C and may be advanced from the first vessel 10 and into the second vessel 12. As the base plate 422 extends into the second vessel 12, the flexible wings 430a, 430b may unfold to a deployed position to engage the sidewall 13 of the second vessel 12. The retention tab 429 may be positioned within the first vessel 10. The first engagement member 410 may then be pushed over the retention tab 429 or the retention member 426 such that the second engagement member 410 is coupled to the first engagement member 410 and tissue of the first vessel 10 and the second vessel 12 is impinged between the base plate 422 of the second engagement member 420 and the plate 412 of the first engagement member 410.

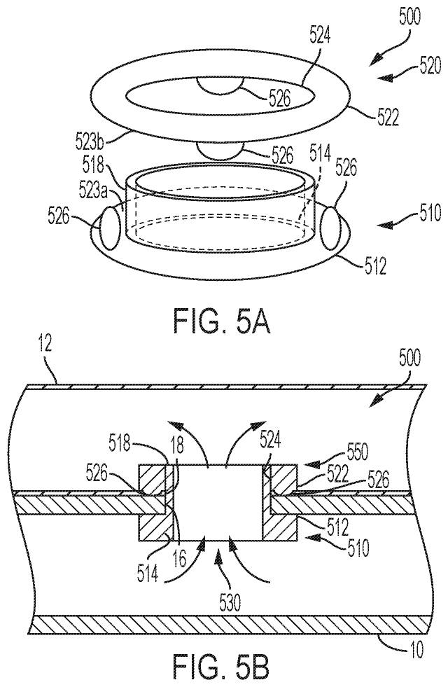

FIGS. 5A and 5B illustrate yet another embodiments of a fistula formation device 500. In the illustrated embodiment, the first engagement member 510 includes a first magnetic disk 512 and the second engagement member 520 includes a second magnetic disk 522. The first magnetic disk 512 defines a first opening 514 therethrough and the second magnetic disk 522 defines a second opening 524 therethrough. Each of the magnetic disks 512, 522 may include one or more magnetic elements, a magnetic core, or the like such that the first magnetic disk 512 is magnetically attracted to the second magnetic disk 522. The first and second magnetic disks 512, 522 are configured to magnetically attract one another such that the first and second openings 514, 524 are axially aligned to provide a flow path from the first vessel 10 to the second vessel 12 as illustrated in FIG. 5B. For example, a conduit 518 may be coupled to one of the first magnetic disk 512 and the second magnetic disc so as to encircle one or more the first opening 514 and the second opening 524. The conduit 518 may be inserted into the other of the first opening 514 and the second opening 524 to provide a flow path between the first vessel 10 and the second vessel 12.

At least one of the first magnetic disk 512 and the second magnetic disk 522 may include one or more spacing elements 526 configured to maintain a separation distance of the first magnetic disk 512 and the second magnetic disk 522. In some embodiments, each of the first magnetic disk 512 and the second magnetic disk 522 include one or more spacing elements 526 (e.g., 1 or more, 2 or more, 3, or more, etc.). The one or more spacing elements 526 may include one or more projections or nubs that extend from an engagement surface 523a, 523b of the at least one of the first magnetic disk 512 and the second magnetic disk 522. The one or more spacing elements 526 may prevent the first and second magnetic disks 512, 522 from applying too strong of a compression to the tissue of the first vessel 10 and the second vessel 12 to prevent degeneration or necrosis of the tissue.

In some embodiments, the one or more spacing elements 526 may include sharp edges configured to cut through tissue of the first vessel 10 and the second vessel 12, such the first magnetic disk 512 directly engages the second magnetic disk 522 via the one or more spacing elements 526. In yet further embodiments, one or more recesses (not shown) formed on the opposite of the first magnetic disk 512 and the second magnetic disk 522 may be configured to receive the one or more spacing elements 526 to prevent sliding of the first magnetic disk 512 relative to the second magnetic disk 522.

The fistula formation device 500 may be delivered via any of the delivery systems and methods described herein (e.g., via first and/or second sheaths and/or with pusher elements removably attached thereto). Referring to FIG. 5B the first magnetic disk 512 may be positioned with the first vessel 10 around an opening 16 (e.g., produced via a needle 112) formed within the first vessel 10 (e.g., via a needle 112). The second magnetic disk 522 may be positioned within the second vessel 12 around the opening 18 formed within the second vessel 12 (e.g., via a needle 112). The magnetic attraction between the first magnetic disk 512 and the second magnetic disk 522 may draw the first and second magnetic disks 512, 522 toward one another such the first opening 514 of the first magnetic disk 512 is disposed around the opening 16 of the first vessel 10 and is axially aligned with the second opening 524 of the second magnetic disk 522 disposed around the opening 18 of the second vessel 12 and the tissue of the first vessel 10 and the second vessel 12 is impinged between the first magnetic disk 512 and the second magnetic disk 522. For example, the magnetic attraction may draw the conduit 518, illustrated as mounted to the first magnetic disk 512, into the second opening 524 of the second magnetic disk 522 such that the conduit 518 passes through (thereby stretching) the opening 16 of the first vessel 10 and the opening 18 of the second vessel 12, and defining a flow path 530 between the first vessel 10 and the second vessel 12. Accordingly the openings 16, 18 in the first vessels 10 and the second vessels 12 are thereby aligned with one another and held in place by the first magnetic disk 512 and the second magnetic disk 522 such that a flow path 530 is defined between the first vessel 10 and the second vessel 12 via the first magnetic disk 512 and the second magnetic disk 522. The first and second magnetic disks 512, 522 may be positioned via any combination of sheaths, pushers, and wires such as described herein, or the like.

When in position, the one or more spacing elements 526 maintain spacing by engaging the opposite magnetic disk either via the tissue compressed between the first magnetic disk 512 and the second magnetic disk 522. In some embodiments, and as described above, the one or more spacing elements 526 may be configured to cut through the tissue of the first vessel 10 and the second vessel 12 and directly engage the other of the first magnetic disk 512 of the second magnetic disk 522.

In the various embodiments, the first and second engagement members of any of the fistula formation devices described herein may be made of any material and/or combination of biocompatible and/or bioabsorbable materials. Example materials may include, but are not limited to, nitinol, stainless steel, titanium, cobalt-chromium alloys, and bioabsorbable polymers. The first and second engagement members may be flexible to allow for traveling through one or more sheaths and/or through the contours of the vessel. In some embodiments, the first and second engagement members may include radiopaque materials and/or markers for visualization by a user, for example, fluoroscopy. In some embodiments, radiopaque materials may be encased with radiolucent materials, such that the first and/or second engagement members include radiopaque cores. Radiopaque materials and or markers may also aid in determining alignment between the first and second engagement members. In some embodiments, the first and/or second engagement embers may be coated or contain drugs. such as, but not limited to, anti-inflammatory, anti-proliferative, and/or anti-platelet agents. Examples include, but are not limited to paclitaxel, sirolimus, and heparin.

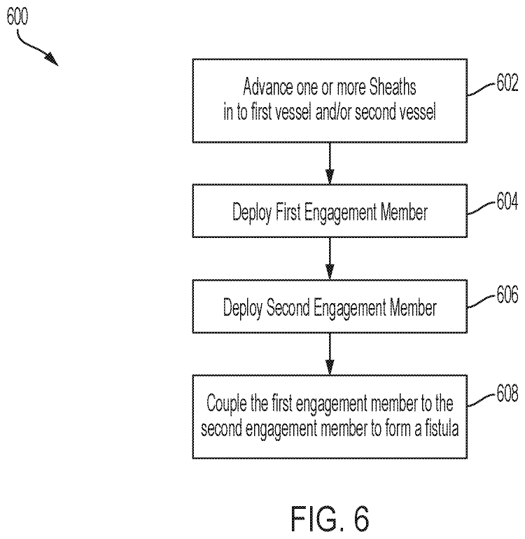

Referring now to FIG. 6 a flow chart depicting a method 600 for forming a fistula is schematically depicted. It is noted that the described method may be used with respect to any of the embodiments described herein unless otherwise noted. In particular, the method includes advancing one or more sheaths into at least one of a first vessel and a second vessel at step 602. For example, and with reference to FIGS. 1B-1F, a first sheath 100 may be advanced within a first vessel 10 and a second sheath 116 may be advanced within a second vessel 12. However, as discussed above, various embodiments may include a single sheath (e.g., sheath 110 or 202) advanced within one of the first vessel 10 and the second vessel 12 to the other of the first vessel 10 and the second vessel 12. In embodiments, a guidewire 114 may be advanced through the one or more sheaths and may provide a path over which components of the fistula formation device may be delivered. That is, the guidewire 114, such as illustrated in FIG. 1B may be advanced from the first sheath 100 into the second vessel into the second sheath 116.

Referring again to FIG. 6, at step 604 the method 600 includes deploying a first engagement member, such as any of the first engagement members described herein, within a first vessel 10 and, at step 606, deploying a second engagement member, such as any of the second engagement members described herein, within the second vessel 12 and deploying the second engagement member within the second vessel at step 606. It is noted that in various embodiments, the first engagement member may instead be deployed in the second vessel 12 and the second engagement member may be deployed in the first vessel 10. For example, and with reference to FIG. 1C, the first engagement member 122 is illustrated as being advanced through the second sheath 116 in the second vessel 12 and the second engagement member 130 is advanced through the first sheath 100 in the first vessel 10. The first a second engagement members 122, 130 are advanced toward one another over the guidewire 114. Referring to FIGS. 3A and 3B, and as further described above, a single sheath 110 may be advanced from the first vessel 10 to the second vessel 12 to deploy to first engagement member 310 (or the second engagement member 320) within the second vessel 12. The sheath 110 may then be withdrawn from the second vessel 12 into the first vessel 10 to deploy the second engagement member 320 (or the first engagement member 310) within the first vessel 10.

Referring again to FIG. 6, at step 608 the method 600 includes coupling the first engagement member to the second engagement member so as to form a fistula between the first vessel 10 and the second vessel 12. That is, the first engagement member and the second engagement member are advanced toward one another to sandwich sidewall tissue of the first and second vessels 10, 12 between the first engagement member and the second engagement member while provide a fluid passage between the first vessel 10 and the second vessel 12. For example, and as illustrated in the above embodiments, coupling the first engagement member to the second engagement member may include inserting one or more retention members, such as described herein, of the second engagement member through the first engagement member to couple the first engagement member to the second engagement member. In some embodiments, such as described above, the first and second engagement members may be magnetic (e.g., magnetic disks 512, 522) such that the first engagement member and the second engagement member are magnetically coupled to one another. Once the first and second engagement members are coupled to one another to form a fistula, the one or more sheaths and/or the guidewire may be removed from the first and second vessels thereby leaving the first and second engagement elements within the first and second vessels to retain a fistula formed and/or retained by the first and second engagement members.

Embodiments of the present disclosure may be described with reference to the following numerical clauses:

1. A fistula formation device, comprising: a first engagement member configured to be positioned within a first vessel, the first engagement member comprising a plate defining a first opening therethrough; and a second engagement member configured to be positioned within a second vessel, the second engagement member comprising: a base plate comprising a second opening extending therethrough; and one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

2. The fistula formation device of any preceding clause, wherein: the plate of the first engagement member comprises a first surface configured to engage a wall of the first vessel and a second surface opposite the first surface; and the one or more retention members are configured to be inserted through the first opening of the first engagement member and comprise one or more retention tabs that engage the second surface of the plate.

3. The fistula formation device of any preceding clause, wherein the one or more retention members comprise a single retention member which circumscribes the second opening.

4. The fistula formation device of any preceding clause, wherein the first engagement member and the second engagement member are bioabsorbable.

5. The fistula formation device of any preceding clause, wherein the one or more retention members comprise a plurality of retention members positioned around and spaced from the second opening.

6. The fistula formation device of any preceding clause, wherein: the plate of the first engagement member comprises: a first surface configured to engage a wall of the first vessel and a second surface opposite the first surface; and a plurality of retention openings axially offset from the first opening; and the plurality of retention members of the second engagement member each comprise one or more retention tabs and are configured to be inserted through the plurality of retention openings and engage the second surface of the first engagement member.

7. The fistula formation device of any preceding clause, wherein the plurality of retention members are configured to pierce through a wall of the first vessel, a wall of the second vessel, and engage the first engagement member.

8. A system for forming a fistula comprising: one or more sheaths configured to be advanced through at least one of a first vessel and a second vessel; and a fistula formation device delivered to the first vessel and the second vessel with the one or more sheaths, the fistula formation device comprising: a first engagement member configured to be positioned within the first vessel, the first engagement member comprising a plate defining a first opening therethrough; and a second engagement member configured to be positioned within the second vessel, the second engagement member comprising: a base plate defining a second opening; and one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

9. The system of any preceding clause, wherein: the one or more sheaths comprise a first sheath configured to be advanced through the first vessel and a second sheath configured to be advanced through the second vessel; the first sheath is configured to deliver the first engagement member to the first vessel; and the second sheath is configured to deliver the second engagement member to the second vessel.

10. The system of any preceding clause, wherein the one or sheaths comprise a single sheath configured to be advanced from the first vessel and into the second vessel, wherein the single sheath is configured to deliver the first engagement member to the second vessel and is configured to be retracted from the second vessel into the first vessel to deliver the second engagement member into the first vessel.

11. The system of any preceding clause, wherein: the plate of the first engagement member comprises a first surface configured to engage a wall of the first vessel and a second surface opposite the first surface; and the one or more retention members are configured to be inserted through the first opening of the first engagement member and comprise one or more retention tabs that engage the second surface of the plate.

12. The system of any preceding clause, wherein the one or more retention members comprise a single retention member which circumscribes the second opening.

13. The system of any preceding clause, wherein the one or more retention members comprise a plurality of retention members positioned around and spaced from the second opening.

14. The system of any preceding clause, wherein: the plate of the first engagement member comprises: a first surface configured to engage a wall of the first vessel and a second surface opposite the first surface; and a plurality of retention openings axially offset from the first opening; and the plurality of retention members each comprise one or more retention tabs and are configured to be inserted through the plurality of retention openings and engage the second surface of the first engagement member.

15. The system of any preceding clause, wherein the plurality of retention members are configured to pierce through a wall of the first vessel, a wall of the second vessel, and engage the first engagement member.

16. A method of forming a fistula, the method comprising: advancing one or more sheaths into at least one of a first vessel and a second vessel; deploying a first engagement member within the first vessel, the first engagement member comprising a plate defining a first opening therethrough; and deploying a second engagement member within the second vessel, the second engagement member comprising: a base plate defining a second opening; and one or more retention members extending from the base plate around the second opening; and coupling the first engagement member to the second engagement member by inserting the one or more retention members of the second engagement member through the plate of the first engagement member to secure the first engagement member to the second engagement member, thereby forming an open conduit through the first engagement member and the second engagement member between the first vessel and the second vessel.

17. The method of any preceding clause, wherein advancing the one or more sheaths into at least one of the first vessel and the second vessel comprises: advancing a first sheath within the first vessel, wherein the first sheath delivers the first engagement member within the first vessel; and advancing a second sheath within the second vessel, wherein the second sheath delivers the second engagement member within the second vessel.

18. The method of any preceding clause, wherein advancing the one or more sheaths into at least one of the first vessel and the second vessel comprises: advancing a single sheath from the second vessel into the first vessel to deploy the first engagement member; and withdrawing the single sheath from the first vessel into the second vessel to deploy the second engagement member within the second vessel.

19. The method of any preceding clause, further comprising: advancing a guidewire through the one or more sheaths from the first vessel to the second vessel; and advancing the first engagement member and the second engagement member toward one another along the guidewire, to sandwich sidewall tissue of the first and second vessels between the first engagement member and the second engagement member.

20. The method of any preceding clause, further comprising removing the guidewire and the one or more sheaths, thereby leaving the first and second engagement members within the first and second vessels to retain the fistula formed by the first and second engagement members.

21. A fistula formation device comprising: a first magnetic disk configured to be positioned within a first vessel, the first magnetic disk defining a first opening therethrough; and a second magnetic disk configured to be positioned within a second vessel, the second magnetic disk defining a second opening therethrough, wherein: each of the first and second magnetic disks are magnetically attracted to one another such that the first opening is axially aligned with the second opening thereby providing a fluid passage between the first vessel and the second vessel; and at least one of the first magnetic disk and the second magnetic disk comprise one or more spacing elements mounted to the at least one of the first magnetic disk and the second magnetic disk and configured to maintain a separation distance of the first magnetic disk from the second magnetic disk.

22. The fistula formation device of any preceding clause, wherein the one or more spacing elements are configured to cut through tissue of the first vessel and the second vessel to directly engage the other of the first magnetic disk and the second magnetic disk.

23. The fistula formation device of any preceding clause, wherein the one or more spacing elements comprise one or more projections extending from an engagement surface of the at least one of the first magnetic disk and the second magnetic disk

24. The fistula formation device of any preceding clause, wherein the first magnetic disk comprises one or more spacing elements and the second magnetic disk comprises one or more spacing elements.

25. The fistula formation device of any preceding clause, further comprising a conduit coupled to one of the first magnetic disk and the second magnetic disk, wherein the conduit is configured to be inserted into one of the first opening of the first magnetic disk and the second opening of the second magnetic disk.

26. A system for forming a fistula, comprising: the fistula formation device of any preceding clause, and one or more sheaths configured to deliver the first and second magnetic disks in to the first vessel and the second vessel.

27. A method of forming a fistula, the method comprising: deploying a first engagement member within the first vessel, the first engagement member comprising a first magnetic disk defining a first opening therethrough; and deploying a second engagement member within the second vessel, the second engagement member comprising a second magnetic disk defining a second opening therethrough, wherein each of the first and second magnetic disks are magnetically attracted to one another such that the first opening is axially aligned with the second opening thereby providing a fluid passage between the first vessel and the second vessel; and at least one of the first magnetic disk and the second magnetic disk comprise one or more spacing elements mounted to the at least one of the first magnetic disk and the second magnetic disk and configured to maintain a separation distance of the first magnetic disk from the second magnetic disk.

28. The method of any preceding clause, wherein the one or more spacing elements are configured to cut through tissue of the first vessel and the second vessel to directly engage the other of the first magnetic disk and the second magnetic disk.

29. The method of any preceding clause, wherein the one or more spacing elements comprise one or more projections extending from an engagement surface of the at least one of the first magnetic disk and the second magnetic disk.

30. The method of any preceding clause, wherein the first magnetic disk comprises one or more spacing elements and the second magnetic disk comprises one or more spacing elements.

31. The method of any preceding clause, wherein one of the first magnetic disk and the second magnetic disk further comprises a conduit configured to be inserted into one of the first opening of the first magnetic disk and the second opening of the second magnetic disk.

It should now be understood that embodiments of the present disclosure are directed to devices, systems, and methods for the formation of a fistula within a body of the subject. Such devices are deployed to form and/or maintain openings formed between two adjacent vessels. For example, devices and methods as provided herein provide an implantable device which may be delivered through a patient's body passages (e.g., vasculature) and used to form a fistula between the body passages. Accordingly, a fistula may be formed with minimal surgical invasiveness into the body. In the embodiments described herein the device includes a first engagement portion and a second engagement portion configured to maintain flow between a first vessel and a second vessel within a body.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A fistula formation device, comprising:
a first engagement member configured to be positioned within a first vessel, the first engagement member comprising a plate defining a first opening therethrough; and
a second engagement member configured to be positioned within a second vessel, the second engagement member comprising:
a base plate comprising a second opening extending therethrough; and
one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

2. The fistula formation device of claim 1, wherein:
the plate of the first engagement member comprises a first surface configured to engage a wall of the first vessel and a second surface opposite the first surface; and
the one or more retention members are configured to be inserted through the first opening of the first engagement member and comprise one or more retention tabs that engage the second surface of the plate.

3. The fistula formation device of claim 1, wherein the one or more retention members comprise a single retention member which circumscribes the second opening.

4. The fistula formation device of claim 1, wherein the first engagement member and the second engagement member are bioabsorbable.

5. The fistula formation device of claim 1, wherein the one or more retention members comprise a plurality of retention members positioned around and spaced from the second opening.

6. The fistula formation device of claim 5, wherein:
the plate of the first engagement member comprises:
a first surface configured to engage a wall of the first vessel and a second surface opposite the first surface; and
a plurality of retention openings axially offset from the first opening; and
the plurality of retention members of the second engagement member each comprise one or more retention tabs and are configured to be inserted through the plurality of retention openings and engage the second surface of the first engagement member.

7. The fistula formation device of claim 5, wherein the plurality of retention members are configured to pierce through a wall of the first vessel, a wall of the second vessel, and engage the first engagement member.

8. A system for forming a fistula comprising:
one or more sheaths configured to be advanced through at least one of a first vessel and a second vessel; and
a fistula formation device delivered to the first vessel and the second vessel with the one or more sheaths, the fistula formation device comprising:
a first engagement member configured to be positioned within the first vessel, the first engagement member comprising a plate defining a first opening therethrough; and
a second engagement member configured to be positioned within the second vessel, the second engagement member comprising:
a base plate defining a second opening; and
one or more retention members extending from the base plate around the second opening, wherein the one or more retention members are configured to extend through the plate of the first engagement member to secure the first engagement member to the second engagement member and form an open conduit through the first engagement member and the second engagement member between the first vessel and the second vessel.

9. The system of claim 8, wherein:
the one or more sheaths comprise a first sheath configured to be advanced through the first vessel and a second sheath configured to be advanced through the second vessel;
the first sheath is configured to deliver the first engagement member to the first vessel; and
the second sheath is configured to deliver the second engagement member to the second vessel.

10. The system of claim 8, wherein the one or sheaths comprise a single sheath configured to be advanced from the first vessel and into the second vessel, wherein the single sheath is configured to deliver the first engagement member to the second vessel and is configured to be retracted from the second vessel into the first vessel to deliver the second engagement member into the first vessel.

11. The system of claim 8, wherein:
the plate of the first engagement member comprises a first surface configured to engage a wall of the first vessel and a second surface opposite the first surface; and
the one or more retention members are configured to be inserted through the first opening of the first engagement member and comprise one or more retention tabs that engage the second surface of the plate.

12. The system of claim 8, wherein the one or more retention members comprise a single retention member which circumscribes the second opening.

13. The system of claim 8, wherein the one or more retention members comprise a plurality of retention members positioned around and spaced from the second opening.

14. The system of claim 13, wherein:
the plate of the first engagement member comprises:
a first surface configured to engage a wall of the first vessel and a second surface opposite the first surface; and
a plurality of retention openings axially offset from the first opening; and
the plurality of retention members each comprise one or more retention tabs and are configured to be inserted through the plurality of retention openings and engage the second surface of the first engagement member.

15. The system of claim 13, wherein the plurality of retention members are configured to pierce through a wall of the first vessel, a wall of the second vessel, and engage the first engagement member.

16. A method of forming a fistula, the method comprising:
advancing one or more sheaths into at least one of a first vessel and a second vessel;
deploying a first engagement member within the first vessel, the first engagement member comprising a plate defining a first opening therethrough; and
deploying a second engagement member within the second vessel, the second engagement member comprising:
a base plate defining a second opening; and
one or more retention members extending from the base plate around the second opening; and
coupling the first engagement member to the second engagement member by inserting the one or more retention members of the second engagement member through the plate of the first engagement member to secure the first engagement member to the second engagement member, thereby forming an open conduit through the first engagement member and the second engagement member between the first vessel and the second vessel.

17. The method of claim 16, wherein advancing the one or more sheaths into at least one of the first vessel and the second vessel comprises:
advancing a first sheath within the first vessel, wherein the first sheath delivers the first engagement member within the first vessel; and
advancing a second sheath within the second vessel, wherein the second sheath delivers the second engagement member within the second vessel.

18. The method of claim 16, wherein advancing the one or more sheaths into at least one of the first vessel and the second vessel comprises:
advancing a single sheath from the second vessel into the first vessel to deploy the first engagement member; and
withdrawing the single sheath from the first vessel into the second vessel to deploy the second engagement member within the second vessel.

19. The method of claim 16, further comprising:
advancing a guidewire through the one or more sheaths from the first vessel to the second vessel; and
advancing the first engagement member and the second engagement member toward one another along the guidewire, to sandwich sidewall tissue of the first and second vessels between the first engagement member and the second engagement member.

20. The method of claim 19, further comprising removing the guidewire and the one or more sheaths, thereby leaving the first and second engagement members within the first and second vessels to retain the fistula formed by the first and second engagement members.

21.-31. (canceled)
